(19) United States
(12) Patent Application Publication
(10) Pub. No.: US 2018/0313805 A1
(43) Pub. Date: Nov. 1, 2018

(54) Method of using a Library of Drug Taggants to Asses Decay in Stored Drug Compositions

(71) Applicants: David R. Hall, Provo, UT (US); Dan Allen, Springville, UT (US); Conrad Rosenbrock, Provo, UT (US); Daniel Hendricks, Provo, UT (US); Andrew Nguyen, Provo, UT (US); Steven J.M. Butala, Provo, UT (US); Travis Niederhauser, Mapleton, UT (US); Joe Fox, Spanish Fork, UT (US); Terrece Pearman, Draper, UT (US)

(72) Inventors: David R. Hall, Provo, UT (US); Dan Allen, Springville, UT (US); Conrad Rosenbrock, Provo, UT (US); Daniel Hendricks, Provo, UT (US); Andrew Nguyen, Provo, UT (US); Steven J.M. Butala, Provo, UT (US); Travis Niederhauser, Mapleton, UT (US); Joe Fox, Spanish Fork, UT (US); Terrece Pearman, Draper, UT (US)

(21) Appl. No.: 15/497,501

(22) Filed: Apr. 26, 2017

(51) Int. Cl. G01N 33/15; G01N 17/00

(52) U.S. Cl. CPC G01N 33/15; G01N 17/004; G01N 17/00

(57) ABSTRACT

We disclose a method of using taggants to assess how and to what extent a drug in a drug composition has decayed in response to environmental conditions and time. The taggants may decay in response to environmental conditions which cause drugs to lose their efficacy. The decay may occur due to improper storage or excursions into certain environmental conditions. These environmental conditions may include light, temperature, moisture, oxidation, and age. By including taggants that have different decay characteristics, the environmental condition that caused the decay may be determined. The amount of time the drug composition was exposed to the environmental condition and the amount of effective drug remaining may also be determined. The disclosed method may reduce the need for a unique assay for each drug to assess decay and determine shelf life. The disclosed method may also be used as a quality control technique for pharmaceutical products.

### BACKGROUND

### Field of the Invention

This disclosure relates to methods of tracking and identifying the age or amount of degradation of pharmaceuticals using drug taggants.

### Background of the Invention

Consumption of drugs which have reduced efficacy or are unsafe due to aging or prolonged exposure to certain conditions creates a health risk. Methods for managing this problem include printing an expiration date on the drug packaging and applying aging strips that change color over time to drug packaging. However, these methods simply predict the status of a consumed drug but do not actually detect the consumption of outdated or degraded drug.

An additional problem is abuse of prescription drugs. If a patient does not consume all the prescribed medication a person other than the patient may consume that medication. This may occur when the patient shares unused drug with another or illegally sells the drug. The drug may not be effective when the other person receives it due to improper storage.

In these and other scenarios it is not only beneficial to be able to identify the consumed drug, but also whether the drug has experienced significant degradation from age, extreme temperature, light, or other environmental conditions.

A method is needed to tag drug compositions in a way that both identifies the drug composition, determines whether it has decayed, how much the drug has decayed, and determines what environmental conditions caused the decay. Such a method may be useful for pharmaceutical manufacturing quality control and confirming efficacy of inventory.

### BRIEF SUMMARY OF THE INVENTION

We disclose a method of using one or more taggants which may be added to drug compositions to assess decay of a drug in the drug composition due to environmental conditions or age. The taggants may have one or more decay characteristics which may include light sensitivity, temperature sensitivity, sensitivity to moisture, and decay due to chemical degradation over time. The drug composition may include taggants that possess qualitatively or kinetically the same decay characteristics as the drug in the drug composition. The drug composition may also include taggants that possess different decay characteristics relative to the drug in the drug composition. Alternatively, multiple taggants, some with the same and some with different decay characteristics may be included.

The taggants may be present in the drug composition in defined ratios. The ratio may provide at least some of the uniqueness of the taggant associated with the drug composition. The taggants, including their relative ratios, may indicate drug composition manufacturer, drug, drug composition, manufacturing batch, dispensing pharmacy, prescribing healthcare provider, healthcare provider's institution, and prescribed user.

Examples of chemicals which may be used as taggants include polyethylene glycol, copovidone, povidone, propylparaben, methyl paraben, acesulfame potassium, mannitol, sorbitol, xylitol, steviol glucuronide, sucralose, oleic acid, trans-anethole, 1, 8-eucalyptol, limonene-2D, linalool, citronellol, riboflavin, tartaric acid, and salts of tartaric acid.

### BRIEF DESCRIPTION OF THE DRAWINGS

FIG. 1A illustrates two taggants prior to applying to a drug capsule.

FIG. 1B illustrates the drug capsule of FIG. 1A after the two taggants have been applied.

FIG. 2 illustrates a method of assessing heat induced drug degradation in a drug composition that includes three taggants which have been applied to the drug composition in defined ratios.

FIG. 3 provides a flow chart which includes steps which may be used in performing an embodiment of a method of using the disclosed drug tagging system to measure drug decay and identify the decay characteristic that caused the decay.

FIG. 4 is a table showing the names and chemical structures of examples of relatively stable molecules which may be used in taggants according to the disclosure.

FIG. 5 is a table showing the names and chemical structures of examples of partially metabolized or degraded molecules which may be used in taggants according to the disclosure.

FIG. 6 shows three variations of povidone which may be used in taggants according to the disclosure.

FIG. 7 is a table showing variations of PEGs which may be used in taggants according to the disclosure.

FIG. 8 provides a flow chart which describes an embodiment of a method of using drug taggants to characterize a rate of drug decay in response to light exposure.

### DETAILED DESCRIPTION OF THE INVENTION

### Definitions

Drug, as used herein, means any pharmacologically active agent or mixture of agents.

We disclose a method of applying taggants to or mixing taggants with drug compositions for later detection of the taggants. The method includes detection of the taggants in a drug composition and assessment of decay of the drug in the drug composition. In some embodiments, multiple taggants are used. In one embodiment, the first taggant includes multiple chemical molecules in a defined ratio. The ratio may be unique to a variety of details about the drug composition including manufacturer, drug, formulation, drug composition, manufacturing batch, dispensing pharmacy, prescribing healthcare provider, healthcare provider's institution, and prescribed user.

Taggants may be sprayed on pills, included as a powder or liquid ingredient in a pill or capsule or dispersed or dissolved in a liquid medication. The taggants may also be dusted on pills, adhered to pills and pills may be dipped in taggant solutions. In an example the taggants are mixed with the drug in powder form before pill or capsule formation so that the taggants are difficult to separate from the active ingredient in the drug composition. Each taggant may be colorized with a distinct visual color or combination of visual colors corresponding to its respective decay characteristics.

The unique drug taggants may be applied to or mixed with the drug composition at a first time period, which may be at the time of manufacture of the drug composition. One or more additional unique drug taggants may be added at a second time period. For example, a first set of unique drug taggants may be added at the time of manufacture of the drug composition in ratios that indicate manufacturer, drug, formulation, drug composition, and manufacturing batch. A second set of unique drug taggants may be sprayed on a group of pills at a second time point. The second set of unique drug taggants may be sprayed on the pills further down the supply chain in ratios that indicate dispensing pharmacy, prescribing healthcare provider, healthcare provider's institution, or prescribed user. Consequently, the pharmacy may spray one or more unique drug taggants on the pills when they accept delivery of the pills. Alternatively, or in addition, the healthcare provider may spray one or more unique drug tags on the pills prior to dispensing them to a patient.

It may be useful to produce pills or other forms of medical formulations which include a placebo instead of an active drug molecule. This may be useful in conducting clinical studies. The disclosed method may be used to confirm that the study protocol was followed and that the patients received the correct pill or medical formulation. Alternatively, the method may be useful to keep those conducting the study blind to which study group a subject is assigned to. The subject's consumption of the drug or placebo may be confirmed after consuming the drug or placebo.

The chemicals included in the taggants may be sensitive to one or more of a variety of conditions. These may include light sensitivity, decay due to temperature sensitivity, decay due to moisture, and decay due to chemical degradation over time. Decay over time may include oxidation over time which may be a result of oxygen exposure. If the sensitivity to specific conditions and the corresponding decay rate (kinetic decay rate) of the taggant is the same or similar to that of the drug in the drug composition, the decay of the drug in the drug composition may be extrapolated to that of the taggants.

In an example, a first chemical is provided in concentration C1 and a second chemical is provided in concentration C2. The first and second chemicals are present in the drug composition in a ratio C2/C1 which represents taggant T21. A third chemical is provided in a concentration of C3 and at a ratio to the second chemical of C3/C1 which represents taggant T31. The drug composition may be stored as inventory over time or subjected to excursions into a variety of environmental conditions. The reduced amounts of chemicals may be represented by C1*, C2*, and C3* which are present in ratios C1*/C2* and C3*/C1*. The decay characteristics of each of the three chemicals is known and may be extrapolated to indicate the amount of drug in the drug composition that has decayed.

In another example, if the decay rates of all taggants are different, with enough taggants, paths in a multidimensional space may be used to categorize the drug even in a decayed or partially decayed state. Taggants with different decay rates may reduce the number of possible distinct taggant ratios as one ratio may degrade into a different ratio, so that the second ratio is not useful. However, since the decay ratios describe a bounded trajectory in the multidimensional dimensional space, classification of the taggant aging status is possible based on the position in the multidimensional space of concentration ratios.

When a drug composition includes multiple drug molecules with multiple taggants, multivariate techniques may be used to estimate the contributing fractions. In this example, taggant ratios of increased orthogonality may be used to improve multivariate discrimination.

In some embodiments, a plurality of taggants may have negligible or similar decay profiles. An additional taggant may be added that has a different decay profile to assist in extrapolating the decay profile of the taggants to that of the drug in the drug composition.

In some embodiments, taggants with qualitatively the same and kinetically similar decay characteristics as the drug may be used. Even if the precise decay rates differ, a mathematical relationship may be used to relate the decay of one compound to another if the relative decay rates are known. In an example, there is a known decay rate for decay of a drug due to age under nominal conditions. The detected decay of the taggant infers that the drug composition has been stored for a period of elapsed time. Consequently, by knowing the elapsed time and decay rate of the drug composition, the amount of active drug remaining may be estimated from the amount of measured drug taggant.

In another example two taggants with different heat sensitivities are included in a drug composition. As one of skill in the art will understand, the reaction rate k1 of a molecule is assumed to be exponentially related to the temperature T by an Arrhenius relationship k1=A1*exp[−E1/(RT)], where R converts temperature to energy units (gas constant), A is frequency factor, and E1 is the activation energy. In this example, the decay of a drug in a drug composition that includes the two taggants is strongly dominated by high temperature excursions. By comparing the degradation fraction of two taggants with different energies it is possible to approximate the time at which the drug was at an elevated temperature and what that temperature was (under the assumption of decay is dominated by a single high temperature excursion or multiple equivalent high temperature excursions). We refer to the two taggants as taggants 1 and 2 and assume to decay independently of each other. The concentrations of taggants 1 and 2 (referred to as C1 and C2 respectively) detected at the time of analysis and normalized to initial concentrations (or presented in a ratio with a highly stable taggant) may be expressed as follows C1(t)=exp(−k1*t), C2(t)=exp(−k2*t). The reaction rates are k1=A1*exp[−E1/(RT)] and k2=A2*exp(−E2/RT). Time may be eliminated from the equations to find a formula for temperature T=(E2−E1)/(R*ln(γ)), where γ=A2*ln(C1)/(A1*ln(C2)). Time may be calculated using the temperature and the exponential decay formula. Then, by knowing the time and the temperature, the decay of the drug may be estimated via its Arrhenius equation.

In some embodiments, the concentration of one or more of the taggants added to the drug composition is approximately the same as the concentration of the drug in the drug composition. The concentration of the unique drug taggants, either individually or jointly, may exceed the concentration of the drug, or drugs, by approximately between 50 percent and 100 percent. This scenario assists in providing quantitative information about the amount of active drug that is present in the drug composition at the time of analysis in addition to its decay profile.

In some embodiments, the decay rate of the taggants in response to specific conditions is approximately the same as the drug in the drug composition. This scenario assists in providing quantitative information about the decay of the drug in the drug composition.

In some embodiments, the drug in the drug composition and at least one of the unique drug taggants possess qualitatively the same decay characteristic as well as kinetically the same decay characteristic. In this example, the decay of the at least one of the unique drug taggants may be directly extrapolated to decay of the drug in response to the environmental condition that triggers the shared decay characteristic.

In some examples, the drug composition may include a third unique drug taggant which is stable in the presence of a variety of environmental conditions. For example, the third unique drug composition may be stable in response to light, temperatures outside a range recommended for storage of the drug, oxygen exposure, moisture, and time. This third unique drug taggant may act as an internal control to correct for loss of taggant due to handling during analysis.

In some embodiments, multiple taggants with different decay sensitivities may be applied to or mixed with the drug composition. For example, one taggant may spontaneously decay over time, one taggant may be sensitive to light exposure, and one taggant may be sensitive to temperatures outside a defined range. When this plurality of taggants is applied to or mixed with a drug composition, the history of the conditions to which the drug composition has been exposed may be determined.

The disclosed method may measure and report or record the detected spectrum or chemical dispersion. A processor may calculate the relative ratios of taggants. Based on the position in a multi-dimensional space of taggant ratios, the amount of decay in one or more axes (for example, age and exposure to heat and light) may be estimated. This calculation may be performed locally on the system or equivalently by a remote processor.

In some embodiments, the taggants are detected using one or more of the following analytical techniques: gas chromatography-mass spectrometry, liquid chromatography, capillary zone electrophoresis with UV absorbance, high performance liquid chromatography with UV absorbance, reverse-phase chromatography, fluorescence spectroscopy, high performance thin layer chromatography, UV spectroscopy, infrared spectroscopy, near IR spectroscopy, mid-IR spectroscopy, visible spectroscopy, nuclear magnetic resonance, ion mobility spectrometry, liquid chromatography-ion mobility spectroscopy, liquid chromatography-electrochemical detection, liquid chromatography-UV spectroscopy with a normal UV photodetector, thin layer chromatography, liquid chromatography, Raman spectroscopy, colorimetric assay, and mass spectrometry. In some embodiments, the taggants are detected in the biological sample using near IR spectroscopy. In some embodiments, the near IR spectroscopy analysis may be conducted using at least one excitation wavelength of between about 1 micron and about 2.5 microns. In some embodiments, spectroscopy analysis may be conducted using at least one excitation wavelength of between about 250 nm and about 800 nm. In some embodiments, the taggants may be measured using colorimetric chemical reactions which may be conducted on a color change chemistry strip.

The taggants may comprise of one or more of polyethylene glycol, copovidone, povidone, propylparaben, methyl paraben, acesulfame potassium, mannitol, sorbitol, xylitol, steviol glucuronide, sucralose, oleic acid, trans-anethole, 1, 8-eucalyptol, limonene-2D, riboflavin, tartaric acid, salts of tartaric acid, linalool, and citronellol. Trans-anethole is a component of anise oil, 1, 8-eucalyptol is a component of eucalyptus oil, and limonene-2D is a component of orange oil. Linalool is a component of coriander oil and citronellol is a component of rose oil or geranium oil. Consequently, the taggants may include anise oil, eucalyptus oil, orange oil, coriander oil, rose oil, and geranium oil.

In embodiments in which polyethylene glycol is included in the taggants, the polyethylene glycol may include polymers with an average molecular weight of between about 400 and about 2000. In some embodiments, the polyethylene glycol included in the taggants may include of one or more of the following average molecular weights: 400, 600, 800, 1000, 1500, and 2000.

In some embodiments in which povidone is included in the taggants, the povidone molecules may be polymers that include 25-mers, 30-mers, 90-mers or a combination thereof.

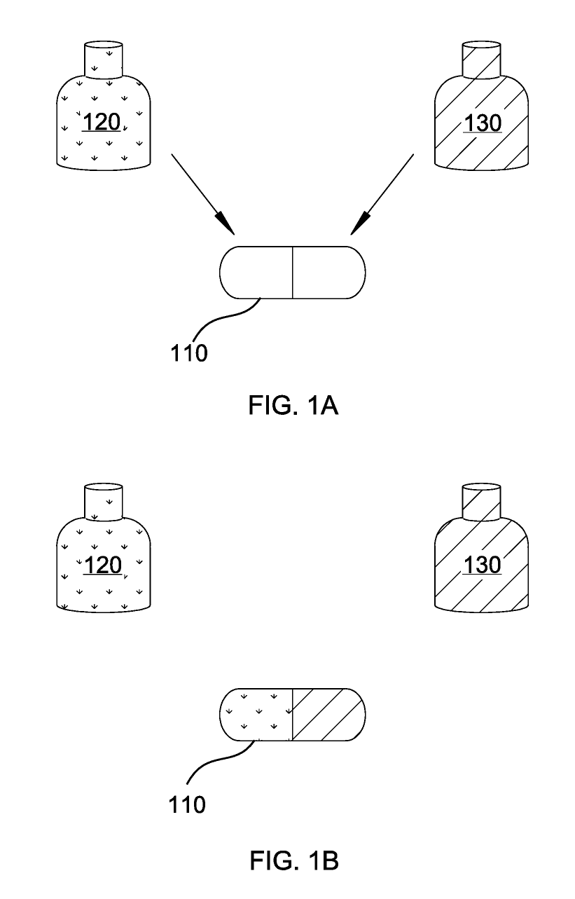

Referring now to the drawings, FIG. 1A illustrate drug capsule 110 which includes a drug composition. Drug capsule 110 has not yet been exposed to a taggant. In this embodiment, two chemicals, chemical 120 and chemical 130 are shown and will be sprayed on the surface of a different half of drug capsule 110 according to the arrows.

FIG. 1B shows drug capsule 110 after chemicals 120 and 130 have been sprayed on its surface. The different shadings on drug capsule 110 correlate with the shadings on the bottles of chemicals 120 and 130.

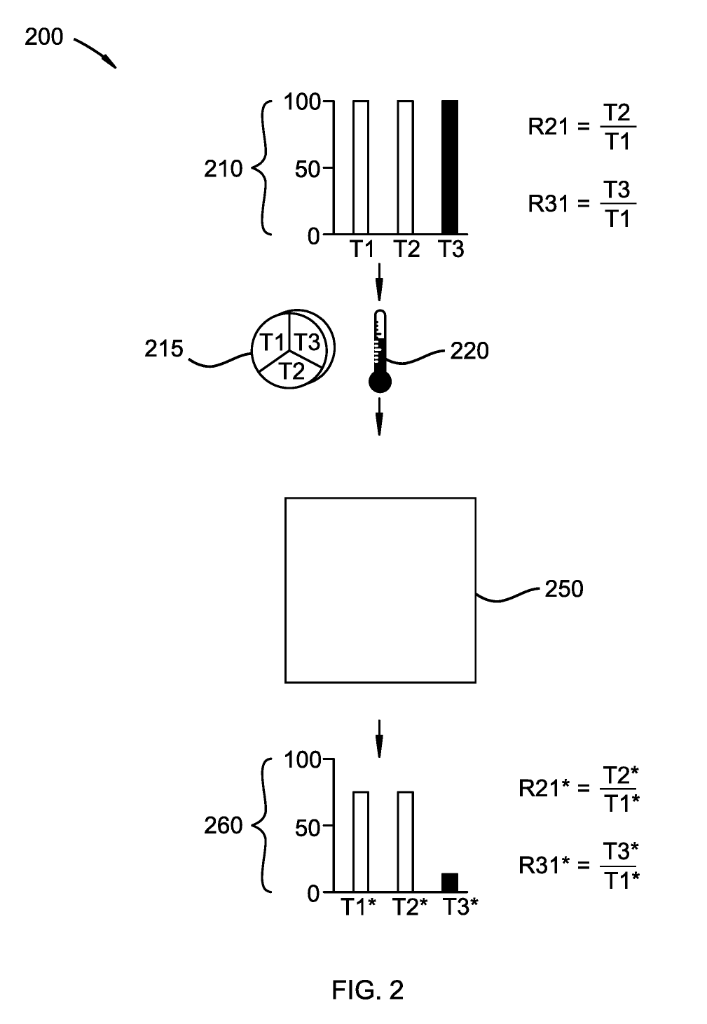

FIG. 2 illustrates a method of using three chemicals as taggants for a drug composition. The three chemicals are T1, T2, and T3 and their original amounts are shown as 100% in bar graph 210. To the right of bar graph 210, the original ratios of chemicals T1, T2, and T3 are shown. The first ratio, R21, is the ratio of T2 to T1. The second ratio, R31, is the ratio of T3 to T1. R21 and R31 represent two taggants. They are unique to other taggants because of the chemicals and because of their relative ratios. The three chemicals have been applied to a drug composition, illustrated as tablet 215 in ratios R21 and R31. In this example, the decay rates of T1 and T2 in response to heat are negligible relative to T3. At some point, tablet 215 is exposed to temperature 220, illustrated in FIG. 2 as a thermometer. Temperature 220 is above the recommended storage temperature for tablet 215 because the drug in tablet 215 is heat sensitive. Consequently, at least one chemical which is similarly heat sensitive is included in the taggants. T3 is especially heat sensitive so failure to detect the original amount of T3 in a user's biological waste indicates heat exposure.

After exposure to heat, the drug composition is analyzed according to the present disclosure using spectrophotometer 250 using near infrared spectrometry. The percentages of the original amounts of T1, T2, and T3 that were applied to tablet 215 at the time of manufacture are measured in the analysis and are found to be reduced. The altered T1, T2, and T3 are presented in bar graph 260 as T1*, T2*, and T3* respectively. The original ratios R21 and R31 are also altered and presented at R21* and R31* respectively. This analysis indicates that the drug in tablet 215 has been exposed to excessive heat and tablet 215 may have contained less active drug at the time of analysis than it did at the time of manufacture.

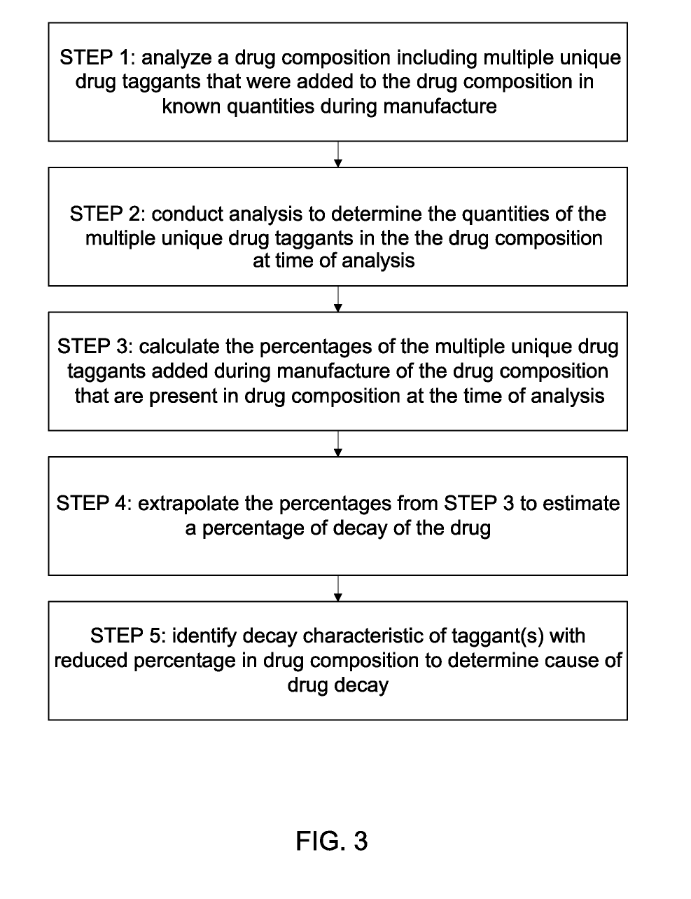

FIG. 3 is a flow chart illustrating a series of steps which may be performed to identify the decay characteristic that caused a drug to decay. In this example, the drug composition includes multiple unique drug taggants which were added to the drug composition in known quantities at the time the drug composition was manufactured. Each of the multiple unique drug taggants has a qualitatively different decay characteristic. At a later time, possibly after the drug composition was stored as inventory for a period of time, a sample of the drug composition is analyzed (Step 1) to identify the unique drug taggants. The unique drug taggants is also quantified (Step 2). The percentage of the original amount of each unique drug taggant that is present in the drug composition at the time of analysis is calculated (Step 3). This information is used to extrapolate to determine the percentage of drug decay (Step 4). The decay characteristics of the particular taggants that are reduced due to decay are known and may be extrapolated to determine what caused the decay of the drug. For example, if the taggant that is light sensitive is greatly reduced, but not other taggants, the drug composition may have been exposed to light resulting in drug decay.

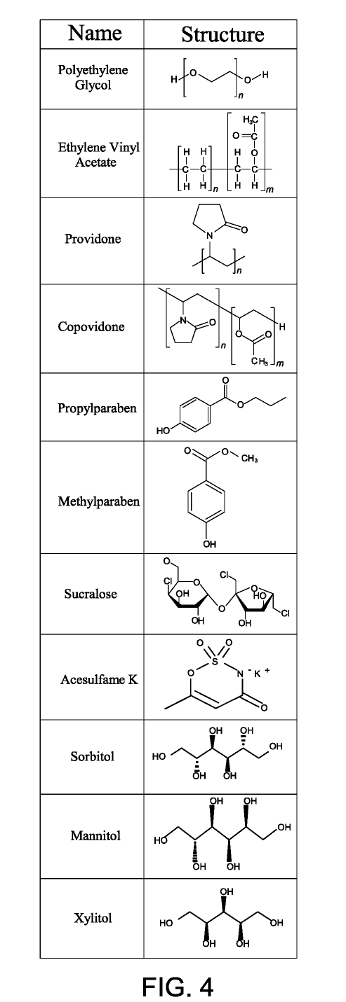

FIG. 4 provides a table which lists examples of chemical entities which may be used in taggants according to the disclosure along with their chemical structures. These include polyethylene glycol, ethylene-vinyl acetate, copovidone, povidone, propylparaben, sucralose, methyl paraben, acesulfame potassium, mannitol, sorbitol, and xylitol. The compounds in the table of FIG. 1 are relatively stable over time and various environmental conditions.

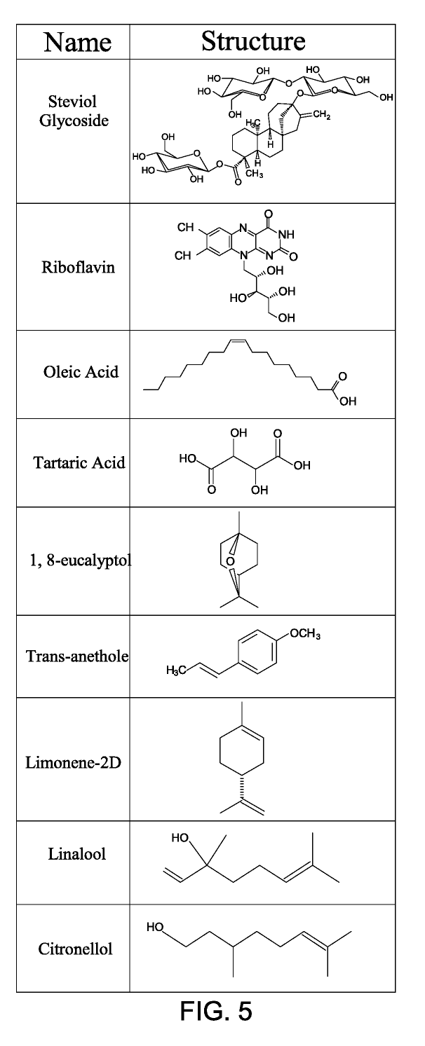

FIG. 5 provides a table which lists examples of chemical entities which may be used in taggants according to the disclosure along with their chemical structures. These include steviol glucuronide, sucralose, oleic acid, trans-anethole, 1, 8-eucalyptol, limonene-2D, riboflavin, tartaric acid, salts of tartaric acid, linalool, and citronellol. Trans-anethole is a component of anise oil, 1, 8-eucalyptol is a component of eucalyptus oil, and limonene-2D is a component of orange oil. Linalool is a component of coriander oil and citronellol is a component of rose oil or geranium oil. Accordingly, anise oil, eucalyptus oil, orange oil, coriander oil, rose oil, and geranium oil may be used in taggants. These chemical entities are relatively unstable over time so may be used to assess the amount of time that has passed after applying the tag to the drug composition or nutritional composition.

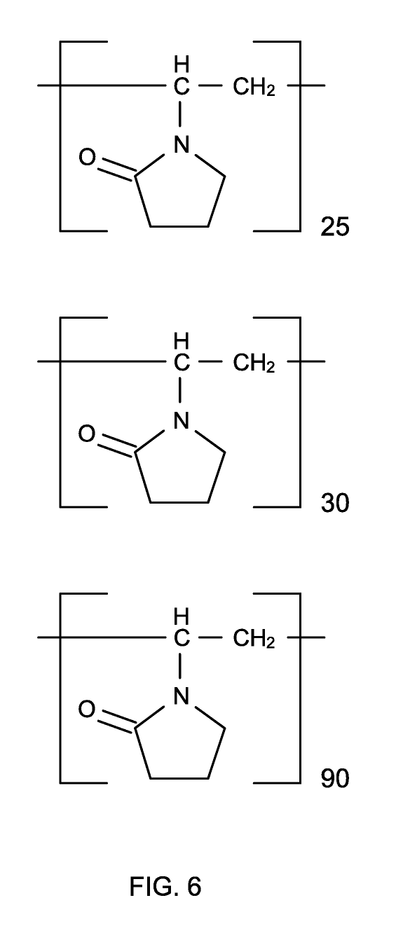

FIG. 6 shows three forms of povidone which may be used in taggants according to the disclosure. These include polymers of varying sizes including a 25-mer, a 30-mer, and a 90-mer.

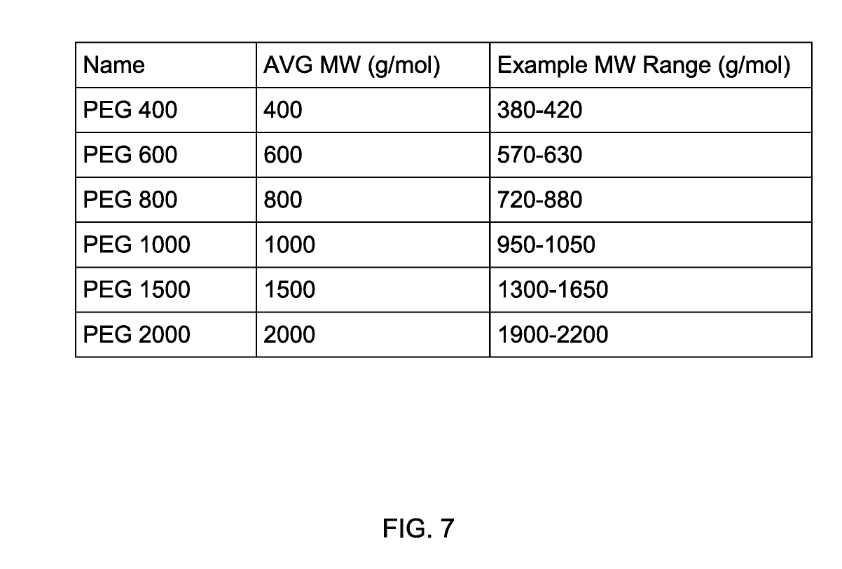

FIG. 7 provides a table which lists examples of polyethylene glycols of varying average molecular weights. These include PEG 400, PEG 600, PEG 800, PEG 1000, PEG 1500, and PEG 2000. Polyethylene glycol is a polymer made of varying numbers of monomers and each polyethylene glycol solution may be comprised of a range of molecular weights, depending on the number of monomers in the polymers, with an average molecular weight.

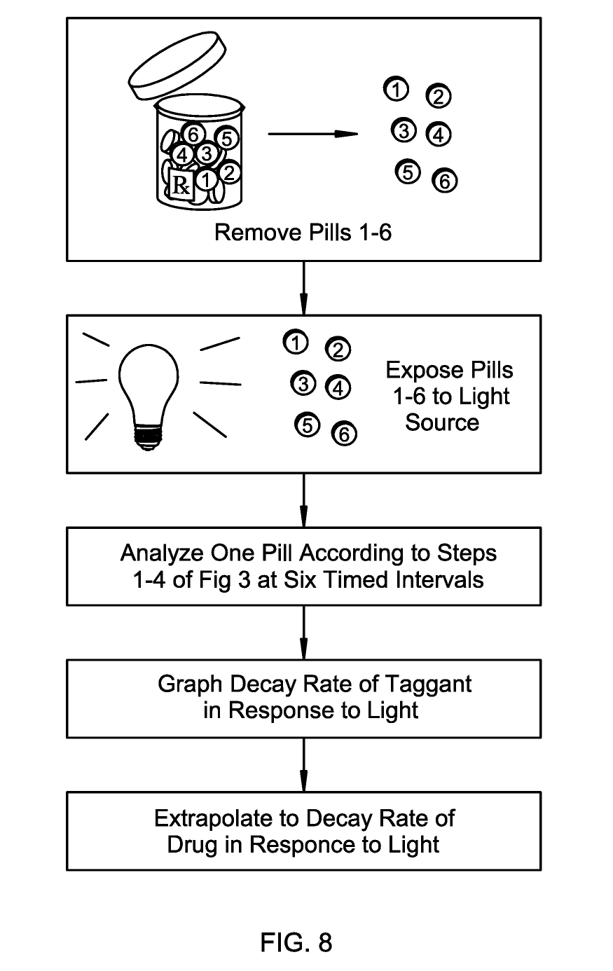

FIG. 8 shows a flow chart which includes steps which may be used to assess the decay characteristic of a drug in a drug composition using the disclosed drug taggants. The first box illustrates a bottle of pills that include pills 1, 2, 3, 4, 5, and 6. Each of pills 1-6 represents a sample of the same drug composition. The drug composition includes taggants as disclosed herein, including at least one taggant that decays in response to light exposure. The bottle of pills is stored for a period of time under defined environmental conditions that include exposure to light. A user wishes to assess the decay rate of the drug in the drug composition in response to light exposure. At timed intervals, one of pills 1-6 is removed from the bottle of pills (leaving the remaining pills exposed to the light source). Each pill is analyzed according to the present disclosure and the percentage of the light sensitive taggant normalized to a stable taggant is calculated. After all of pills 1-6 have been analyzed, the percentages are graphed and the decay rate of the light sensitive taggant is determined. This decay rate is then extrapolated to calculate the decay rate of the drug in the drug composition as disclosed herein.

While specific embodiments have been illustrated and described above, it is to be understood that the disclosure provided is not limited to the precise configuration, steps, and components disclosed. Various modifications, changes, and variations apparent to those of skill in the art may be made in the arrangement, operation, and details of the methods and systems disclosed, with the aid of the present disclosure.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art that changes may be made to the details of the above-described embodiments without departing from the underlying principles of the disclosure herein.

We claim:

1. A method of detecting drug taggants in a drug composition to assess drug decay comprising the steps of:
a) analyzing a drug composition to detect a signal produced by a first and a second unique drug taggant, wherein the drug composition comprised the following in known quantities and in a first ratio at a first time point: at least one drug or placebo, and the first and the second unique drug taggants;
b) calculating a second ratio by dividing the signal produced by the first unique drug taggant by the signal produced by the second unique drug taggant; and
c) extrapolating a difference between the first ratio and the second ratio to determine a fraction of decayed drug, wherein the first unique drug taggant comprises a first decay characteristic, and wherein the second unique drug taggant comprises a second decay characteristic, wherein the at least one drug comprises at least one of the first and the second decay characteristics, and wherein the first decay characteristic is detectably different from the second decay characteristic.

2. The method of claim 1, wherein the at least one drug consists of one drug, wherein the one drug comprises the first and the second decay characteristics.

3. The method of claim 1, wherein the first and second decay characteristics are qualitatively the same and kinetically different.

4. The method of claim 1, wherein steps a) through c) are repeated a plurality of times after exposure of the drug composition to a condition that induces decay of the drug in response to the first decay characteristic, and further comprising the step of determining a decay rate of the drug in response to the condition that induces decay of the drug in response to the first decay characteristic.

5. The method of claim 1, wherein the first ratio is indicative of one of the following: drug composition manufacturer, at least one drug, drug composition, manufacturing batch, dispensing pharmacy, prescribing healthcare provider, healthcare provider's institution, and prescribed user.

6. The method of claim 1, wherein at least one additional unique drug taggant is added at a second time period.

7. The method of claim 1, wherein the drug composition further comprises an additional plurality of unique drug taggants, wherein the additional plurality of unique drug taggants are added to the drug composition in known quantities and in a plurality of ratios, and wherein each of the plurality of ratios is indicative of one of the following: drug composition manufacturer, at least one drug, drug composition, manufacturing batch, dispensing pharmacy, prescribing healthcare provider, healthcare provider's institution, and prescribed user.

8. The method of claim 1, wherein the first unique drug taggant and the second unique drug taggant are independently selected from the following: polyethylene glycol, copovidone, povidone, propylparaben, methyl paraben, acesulfame potassium, mannitol, sorbitol, xylitol, steviol glucuronide, sucralose, oleic acid, trans-anethole, 1, 8-eucalyptol, limonene-2D, riboflavin, tartaric acid, salts of tartaric acid, linalool, and citronellol.

9. The method of claim 8, wherein either the first unique taggant, the second unique taggant, or both the first and second unique taggants consists of polyethylene glycol, and wherein the polyethylene glycol comprises polymers of one or more of the following average molecular weights: 400, 600, 800, 1000, 1500, and 2000.

10. The method of claim 8, wherein either the first unique drug taggant, the second unique drug taggant, or both the first and second unique drug taggants consist of polyethylene glycol, and wherein the polyethylene glycol comprises polymers with an average molecular weight of between about 400 and about 2000.

11. The method of claim 8, wherein either the first unique drug taggant, the second unique drug taggant, or both the first and second unique drug taggants consists of povidone molecules, and wherein the povidone molecules consist of one or more of the following number of monomers: 25, 30, and 90.

12. The method of claim 1, wherein the signals produced by the first taggant and the second taggant are detectable using one or more of the following analytical techniques: gas chromatography-mass spectrometry, liquid chromatography, capillary zone electrophoresis with UV absorbance, high performance liquid chromatography with UV absorbance, reverse-phase chromatography, fluorescence spectroscopy, high performance thin layer chromatography, UV spectroscopy, infrared spectroscopy, near IR spectroscopy, mid-IR spectroscopy, visible spectroscopy, nuclear magnetic resonance, ion mobility spectrometry, liquid chromatography-ion mobility spectroscopy, liquid chromatography-electrochemical detection, liquid chromatography-UV spectroscopy with a normal UV photodetector, thin layer chromatography, liquid chromatography, Raman spectroscopy, colorimetric assay, and mass spectrometry.

13. The method of claim 1, wherein the first decay characteristic is qualitatively different from the second decay characteristic, and wherein each of the first and second decay characteristics are independently selected from the following: decay due to light sensitivity, decay due to temperature sensitivity, decay due to oxidation, decay due to moisture content, and decay due to chemical degradation over time.

14. The method of claim 13, further comprising the steps of:
a) comparing the first and the second ratios;
b) determining which of the first and second unique drug taggants are reduced in the biological sample relative to the first time point;
c) extrapolating a decay characteristic from the first or second unique drug taggant found to be reduced in step b) to a cause of decay of the drug.

15. The method of claim 1, wherein a decay rate of the first taggant in response to the first decay characteristic is approximately the same as a decay rate of the drug in response to the first decay characteristic.

16. The method of claim 1, wherein the drug composition comprises a plurality of drugs and further comprising the step of applying a multivariate technique to identify a cause of decay for each of the plurality of drugs.

17. The method of claim 1, wherein a concentration of the first unique drug taggant, a concentration of the second unique drug taggant, or the concentration of both the first and the second unique drug taggants is approximately the same as a concentration of the drug in the drug composition.

18. The method of claim 1, wherein the drug composition further comprises a third unique drug taggant, wherein the third unique drug taggant is stable relative to the first and second unique drug taggants in response to light, temperatures outside a range recommended for the drug, oxygen exposure, moisture content, and time.

19. The method of claim 18, wherein the signals produced by the first and the second unique drug taggants in the drug composition are normalized to a signal produced by the third unique drug taggant in the drug composition.

20. The method of claim 1, wherein the concentration of the unique drug taggants, either individually or jointly, exceed the concentration of the drug or placebo by approximately between 50 percent and 100 percent.
